(19) United States
(12) Patent Application Publication
(10) Pub. No.: US 2012/0022033 A1
(43) Pub. Date: Jan. 26, 2012

(54) Methods for decreasing cardiovascular risk in postmenopausal women

(75) Inventors: Stephen Simes, Long Grove, IL (US); Michael Snabes, Winnetka, IL (US); Joanne Zborowski, Lake Zurich, IL (US)

(73) Assignee: BIOSANTE PHARMACEUTICALS, INC., Lincolnshire, IL (US)

(21) Appl. No.: 13/135,999

(22) Filed: Jul. 20, 2011

Related U.S. Application Data
(60) Provisional application No. 61/400,205, filed on Jul. 23, 2010.

(51) Int. Cl. A61K 31/56; A61P 9/00; A61K 31/35

(52) U.S. Cl. 514/178; 514/453

(57) ABSTRACT

Methods for decreasing the risk of cardiovascular events in postmenopausal women having a high risk for a cardiovascular event are provided. In particular, methods for decreasing the risk of cardiovascular events in postmenopausal woman at high risk for cardiovascular events by administering to the woman a therapeutically effective amount of an androgen, whereby administering the androgen decreases the risk of cardiovascular events in the woman compared to untreated postmenopausal woman at high risk for cardiovascular events are provided.

This application claims priority from U.S. Provisional Application Ser. No. 61/400,205, filed Jul. 23, 2010. The entirety of this provisional application is hereby incorporated by reference.

### FIELD OF THE INVENTION

The present invention relates to methods for decreasing the risk of a cardiovascular event in postmenopausal women having a high risk for cardiovascular events. In particular, the present invention relates to methods for decreasing the risk of cardiovascular events in postmenopausal women at high risk for cardiovascular events by administering to the women a therapeutically effective amount of an androgen, whereby administering the androgen decreases the risk of cardiovascular events in the women compared to untreated postmenopausal women at high risk for cardiovascular events.

### BACKGROUND

Cardiovascular disease is the leading cause of death in women. Compared to men, premenopausal women are relatively protected from cardiovascular disease, but gradually lose this protection following menopause as estrogen and possibly androgen levels decline. Patterns in Coronary Heart Disease—Morbidity and Mortality in the Sexes: A 26-Year Follow-Up of the Framingham Population, Lerner & Kannel, Amer. Heart J., 111: 383-90 (1986). The onset of cardiovascular disease is hastened in women by prematurely induced surgical menopause and its attendant reduction in endogenous hormone levels. Time Interval from Castration in Premenopausal Women to Development of Excessive Coronary Atherosclerosis, Parrish, H. M. et al., Amer. J. Obst. Gynecol., 99: 155-62 (1967).

Following menopause, the rate of cardiovascular disease in women, such as hyperlipidemia, increases to match the rate seen in men. In the eighth and ninth decades, the occurrence of deaths from ischemic heart disease, approaches that of men (Havlik, R. J. and Manning-Feinleid, P. H. 1979, NIH Publication No. 79-1610, U.S. Department of HEW). It has been reported in the literature that postmenopausal women undergoing estrogen therapy have a return of serum lipid levels to concentrations similar to those of the premenopausal state. Despite this normalization of serum lipids, estrogen does not translate to fewer cardiovascular events (Hsia, J., et al. Arch Intern Med. 2006;166(3):357-65; Hodis, H. N. and Mack, W. J. Menopause. 2007). Indeed estrogen is not recommended as a cardio-protectant.

To date the effect of androgens on cardiovascular events in postmenopausal women has not been investigated with the scientific rigor required to resolve this issue including the need for a sufficient duration of exposure or a sufficiently large study population. However the results of a late stage, randomized clinical trial of a precise dose of a transdermal testosterone formulation to postmenopausal women at high risk for cardiovascular events significantly reduced the number of cardiovascular events compared to the expected number of cardiovascular events for an untreated postmenopausal woman.

Therefore, the formulations and methods of the present invention may be used to reduce the occurrence of cardiovascular events in postmenopausal women.

### SUMMARY OF THE INVENTION

In one aspect of the invention, a method for decreasing the risk of a cardiovascular event in a postmenopausal woman at high risk for cardiovascular events, comprising: administering to the woman a formulation comprising a therapeutically effective amount of an androgen, whereby administering the formulation decreases the risk of cardiovascular events in the woman compared to an untreated postmenopausal woman at high risk for cardiovascular events is provided.

In certain aspects of the invention, the woman is surgically postmenopausal.

In other aspects of the invention, the woman is naturally postmenopausal.

In some embodiments, the androgen is selected from the group consisting of testosterone (17-β-hydroxyandrostenone), testosterone enanthate, testosterone propionate, testosterone decanoate, testosterone cypionate, methyl testosterone, testolactone, oxymetholone, fluoxymesterone and enanthate, propionate, cypionate, phenylacetate, acetate, isobutyrate, buciclate, heptanoate, decanoate, undecanoate, caprate and isocaprate esters of testosterone and 4-dihydrotestosterone. In a preferred embodiment, the androgen is testosterone, more preferably, present in an amount between about 0.50 mg to about 2.4 mg and even more preferably, between about 2.0 and 2.5 mg. In another preferred embodiment, the amount of testosterone in the formulation is 2.2 mg. In certain embodiments, the formulation may be delivered once per day, and delivers to the woman a nominal daily, therapeutically effective dose of 300 micrograms of testosterone per day.

In certain embodiments, the androgen is administered in accordance with the methods conveniently in the form of an oral dose formulation.

In certain embodiments, the androgen, preferably testosterone, is administered in accordance with the methods conveniently in the form of a transdermal or transmucosal formulation, and the transdermal and transmucosal formulation may be provided in the form of a gel, lotion, cream, ointment, emulsion, or suspension.

In certain embodiments, the androgen transdermal formulation comprises at least one of a polyalcohol, alkanol, permeation enhancer, gelling agent, neutralizing agent, buffering agent, moisturizing agent, humectant, surfactant, antioxidant, emollient, or buffer.

In certain preferred embodiments, the transdermal formulation comprises an amount of testosterone between about 0.50 mg to about 2.4 mg, more preferably, 2.2 mg, and about 30% to about 98% ethanol or isopropanol; about 0.1% to about 5% isopropyl myristate; about 1% to about 5% sodium hydroxide; and about 0.1% to about 5% of a gelling agent (by weight of the formulation), or comprises about 50% to about 75% ethanol; about 0.5% to about 2% isopropyl myristate; about 1% to about 3% sodium hydroxide; about 0.5% to about 2% polyacrylic acid; and water in an amount sufficient to make the formulation 100% (by weight of the formulation).

In still other preferred embodiments, the testosterone formulation can further include an alkanol, for example, a C₂ to C₄ alcohol such as ethanol, isopropanol, and/or n-propanol, in an amount between about 5 to 80%; a polyalcohol such as polypropylene glycol in an amount between about 1% to 30%, preferably 6%,; and a permeation enhancer, such as diethylene glycol monomethyl ether or diethylene glycol monoethyl ether in an amount between about 1 to 30% by weight, preferably 5%. The gel formulation in accordance with the invention facilitates the absorption of testosterone by the subject's dermal or mucosal surfaces, and minimizes the transfer or removal of the formulation from the user's skin after application.

The alkanol is provided in combination with water to form a hydroalcoholic mixture. Preferably, the alkanol comprises about 5% to 80% and the water comprises about 20% to 95% of the mixture by weight. The hydroalcoholic mixture may be present in an amount of about 40 to 98%, more preferably 47.5%, by weight of the formulation.

In another preferred embodiment, the androgen, preferably testosterone, is present in an amount of about 0.50 mg to about 2.4 mg, more preferably, 2.2 mg, in a formulation containing: a fatty acid percutaneous absorption promoter in an amount of between 0.1% and 20% (w/w), preferably capric acid, lauric acid, myristic acid, palmitic acid, stearic acid, oleic acid, palmitoleic acid, linoleic acid or linolenic acid, ethanol or isopropanol between 10% and 90% (w/w), and a stabilizer comprising the fatty acid ester of the fatty acid and the alcohol and present in an amount of between 0.1% and 10% (w/w), preferably ethyl oleate, isopropyl oleate, isopropyl myristate, isopropyl palmitate, ethyl octanoate, ethyl dodecanoate, ethyl linoleate or ethyl linolenate.

This formulation may further comprise between 0.5% and 20% of a polyalcohol (w/w), preferably glycerol, propylene glycol, polyethylene glycol, or mixtures thereof. The androgen formulation may further comprise between 0.2% and 30% (w/w) of a gelling agent. In preferred embodiments, the gelling agent is selected from carbomers, non-preneutralized acrylic polymers, cellulose derivatives, poloxamers, poloxamines, chitosan, dextran pectins, natural gums, and mixtures thereof. The formulation may also include a neutralizer. In preferred embodiments, the neutralizer is selected from triethanolamine, sodium hydroxide, ammonium hydroxide, potassium hydroxide, arginine, aminomethylpropanol and tromethamine, and mixtures thereof.

In other embodiments, the formulation comprises on a weight basis: (a) from about 1 to about 12% octyl salicylate; (b) from about 45 to about 90% of an alkanol selected from the group consisting of ethanol, isopropanol, and a mixture thereof; (c) from about 5 to about 45% water; and (d) from about 0.5 to about 5% of a gelling agent.

In still other embodiments, the formulation has pH of about 4 to about 8 and consists essentially of (i) permeation enhancer, preferably oleic acid; (ii) a C₁ to C₄ alkanol; and (iii) a polyalcohol; and iv) a gelling agent. In one embodiment, the alcohol is a member selected from the group consisting of ethanol, propanol, isopropanol and mixtures thereof, and is present from about 5% to about 65% weight to weight of the formulation, more preferably, 10% to about 40% weight to weight of said composition, and even more preferably, 25% to about 35% weight to weight of the formulation. In certain preferred embodiments, the polyalcohol is ethylene glycol, butylene glycol or propylene glycol, and the gelling agent is selected from Carbopol 1342, Carbopol 940, Klucel and Klucel HF, and is present at about 0.1% to about 10%.

In accordance with a further aspect of the invention, the androgen formulations for use in the methods are delivered using a transdermal patch or applied as an aerosol, as a spray. In certain embodiments the transdermal patch is a matrix-type transdermal patch or a liquid reservoir patch

In accordance with a still further aspect of the invention, the androgen formulations for use in the methods can be dispensed from a metered dosage device to provide convenience as well as precise metered dosages to users. Accordingly, the metered dosage device can be configured to dispense a precise amount of the androgen formulation which corresponds to a desired and prescribed dosage of androgen to the user. In preferred embodiments, the androgen is testosterone and formulated to deliver to the woman a nominal daily, therapeutically effective dose of 300 micrograms of testosterone per day.

In certain embodiments, the decrease in the risk of a cardiovascular event in the treated women compared to the risk of cardiovascular events in untreated women is at least 10%, at least 20%, at least 30%, at least 40%, at least 50%, at least 60%, at least 70%, at least 75%, at least 80%, at least 85%, at least 90%, at least 95%, or at least 99%.

### DETAILED DESCRIPTION OF THE INVENTION

The present invention relates to methods for decreasing the risk of cardiovascular events in postmenopausal women at high risk for cardiovascular events. In particular, the present invention relates to methods for decreasing risk of cardiovascular events in high risk postmenopausal women, comprising: administering to the women a formulation comprising a therapeutically effective amount of an androgen that reduces the occurrence of cardiovascular events in the women compared to an untreated postmenopausal women.

The invention is predicated, in part, upon the unexpected findings from a US-based, multi-center, randomized, placebo-controlled Phase 3 clinical trial, where the administration of a precise amount of testosterone in a transdermal formulation to postmenopausal women at high risk for cardiovascular events reduced the number of cardiovascular events compared to the expected number of cardiovascular events for an untreated postmenopausal woman. The findings indicate an unanticipated lower than expected cardiovascular event rate in postmenopausal woman using testosterone for the treatment of cardiovascular events.

### Definitions

Unless defined otherwise, all technical and scientific terms used herein have the same meaning as is commonly understood by one of skill in the art to which this invention belongs. All patents and publications referred to herein are incorporated by reference.

As used herein, a “cardiovascular event” or “cardiovascular events” means one of the following occurrences: a cardiovascular death, a non-fatal stroke, a non-fatal myocardial infarction, hospitalized unstable angina (including acute coronary syndrome), angioplasty, coronary bypass surgery, a pulmonary embolism and deep vein thrombosis.

As used herein, “dose” and “dosage” mean a specific amount of active or therapeutic agents for administration.

As used herein, “high risk of cardiovascular events” means a woman has a minimum of a 2-point cardiovascular disease risk factor as determined by the following point scale: 1) age 60 to less than 70 years (1 point), 2) 70 years or greater (2 points), 3) diabetes mellitus (2 points), 4) presently smoking at least 10 cigarettes/day (or the equivalent, e.g. uses chew tobacco a minimum of 2 hours daily) (1 point), 5) blood pressure (1 point): systolic≧150 mmHg and/or diastolic≧95 mmHg (based on two readings taken at least 30 minutes apart) and/or taking antihypertensive medications (for treatment of hypertension), 6) dyslipidemia (1 point): LDL>160 mg/dl and/or HDL<45 mg/dl and triglycerides>250 mg/dl and/or taking lipid-lowering medication (over-the-counter products are not considered to be acceptable treatment forms), 7) ankle-brachial index<0.6 (2 points), 8) documented history of cardiovascular disease, i.e., myocardial infarction, stroke, hospitalization for unstable angina/acute coronary syndrome, revascularization of the coronary, carotid, or peripheral circulations.

As used herein, “naturally postmenopausal woman” means a woman who has not had a menstrual period for at least 12 months.

As used herein, “mucosa” means any moist anatomical membrane or surface on a mammal such as oral, buccal, vaginal, rectal, nasal or ophthamalic surfaces

As used herein, “oral administration” means swallowing, chewing, or sucking of an oral dosage form comprising the therapeutically effective amount of the androgen.

As used herein, “surgically menopausal women” means women who have undergone bilateral oophorectomy.

As used herein, “therapeutically effective amount of an androgen” means a sufficient amount of an androgen to provide the desired therapeutic effect to prevent the occurrence or reduce the number of cardiovascular events in the woman administered the androgen.

As used herein, “transdermal formulation” means a formulation for transdermal administration i.e., delivery by passage of an androgen through the skin and into the bloodstream.

As used herein, “transmucosal formulation” means a formulation for transmucosal administration i.e., delivery by passage of an androgen through the mucosal tissue and into the bloodstream.

### Androgens

There are a number of exemplary androgens that are suitable for use in the methods disclosed herein. Examples of androgens which may be used in this invention include testosterone (17-.beta.-hydroxyandrostenone), and testosterone esters, such as testosterone enanthate, testosterone propionate, testosterone decanoate and testosterone cypionate. The aforementioned testosterone esters are commercially available or may be readily prepared using techniques known to those skilled in the art or described in the pertinent literature. Also, pharmaceutically acceptable esters of testosterone and 4-dihydrotestosterone, typically esters formed from the hydroxyl group present at the C-17 position (such as enanthate, propionate, cypionate, phenylacetate, acetate, isobutyrate, buciclate, heptanoate, decanoate, undecanoate, caprate and isocaprate esters); and pharmaceutically acceptable derivatives of testosterone such as methyl testosterone, testolactone, oxymetholone and fluoxymesterone may be used.

In a preferred embodiment, the androgen is testosterone, more preferably, present in an amount between about 0.50 mg to about 2.4 mg and even more preferably, in an amount between about 2.0 and 2.5 mg. In another preferred embodiment, the amount of testosterone in the formulation is 2.2 mg. In certain embodiments, the androgen is formulated for oral administration or for transmucosal administration. In certain other embodiments, the formulation may be delivered once-a-day in an amount of about 0.22 gr of a transdermal formulation that may optionally be delivered using a transdermal patch or applied as an aerosol, as a spray.

### Oral Formulations

The therapeutically effective amount of the androgen can be administered through oral dosage forms such as liquids, synthetic oils, oral suspensions, jellies, gum drops, elixirs, syrups, capsules, caplets, troches or tablets. Any formulation which can administer a therapeutically effective dosage form orally is contemplated including immediate release and sustained release dosage forms. Often these formulations can also include viscosity modifiers, surfactants, preservatives, solubilizing agents, microcapsules, microparticles, granules, diluents, binders, fillers, lubricants, colors, flavors and the like.

Oral formulations for delivery of androgens may be manufactured by conventional techniques used in the art of the pharmaceutical industry and are well known to those skilled in the art (e.g., see U.S. Pat. Nos. 6,096,338; 6,517,870; 6,652,880; 6,696,482, 6,733,796; 7,097,851; 7,138,389; and 7,201.913). In the case of solid formulations, the formulation can be prepared, for example, by extruding granulation methods, crushing granulation methods, dry granulation methods, fluidized bed granulation methods, tumbling granulation methods, high shear mixing granulation methods, wet compression methods, direct compression methods and the like.

In addition, enteric coatings may be provided as part of the solid formulation. The enteric coating may be an essentially conventional coating material known for enteric coating, for example, enteric polymers such as cellulose acetate phthalate, cellulose acetate succinate, methylcellulose phthalate, ethylhydroxycellulose phthalate, polyvinylacetate phthalate, polyvinylbutyrate acetate, vinyl acetate-maleic anhydride copolymer, styrene-maleic mono-ester copolymer, methyl acrylate-methacrylic acid copolymer, methacrylate-methacrylic acid-octyl acrylate copolymer, etc. These may be used either alone or in combination, or together with other polymers. The enteric coating may also include insoluble substances which are neither decomposed nor solubilized in living bodies, for example, alkyl cellulose derivatives such as ethyl cellulose, crosslinked polymers such as styrene-divinylbenzene copolymer, polysaccharides having hydroxyl groups such as dextran, cellulose derivatives which are treated with bifunctional crosslinking agents such as epichlorohydrin, dichlorohydrin, 1,2,3,4-diepoxybutane, etc. The enteric coating may also include starch and/or dextrin. The enteric coating may also include an anti-tack agent such as talc, silica, or glyceryl monostearate. The enteric coating can also contain other ingredients such as surfactants, pigments, and fillers. The enteric coating may be formed as a single layer, or as multiple layers. If the coating includes multiple layers, then compositions of the layers may be the same or different, and may be of the same or varying thicknesses.

Alternatively, a liquid formulation for oral administration comprising the androgen may be used. In one embodiment, the androgen, preferably testosterone or testosterone undecanoate, is formulated in a liquid pharmaceutical composition consisting essentially of a glyceride of a long chain fatty acid and a lipophilic surfactant; and wherein the liquid composition is substantially free from free fatty acid. In certain embodiments, the glyceride of a long chain fatty acid is present in an amount from about 15% to about 70% by weight in the liquid composition, and the lipophilic surfactant is present in an amount from about 30% to about 60% by weight in the liquid composition. In certain preferred embodiments, the long chain triglyceride is selected from the group consisting of arachis oil, soya bean oil, castor oil, corn oil, safflower oil, olive oil, apricot kernel oil and sesame oil, and combinations thereof.

### Transdermal and Transmucosal Formulations

In certain embodiments, a therapeutically effective amount of an androgen is administered to a postmenopausal woman by transdermal or transmucosal application. A number of formulations for transdermal or transmucosal administration of androgens are suitable for use in the methods herein, and are well known to those of skill in the art (e.g., see U.S. Pat. Nos.: 5,906,830; 6,228,852; 6,348,210; 6,503,894; 6,562,369; 6,562,370; 6,743,448; 7,018648; 7,198,801; and 7,611,727).

In general, the transdermal and transmucosal formulations comprise an androgen and delivery vehicle that facilitates penetration and absorption of the androgen. In particular, the formulation comprises an androgen; and a delivery vehicle, which may comprise an alkanol, a polyalcohol, and a permeation enhancer to provide permeation enhancement of the androgen through mammalian dermal or mucosal surfaces. The formulation may further comprise a gelling agent, neutralizing agent, buffering agent, moisturizing agent, humectant, surfactant, antioxidant, emollient, and/or buffer, and may be provided in the form of a gel, lotion, cream, ointment, emulsion, or suspension.

Polyalcohols. In accordance with the invention, the polyalcohol may be advantageously present in an amount between about 1% and 30% by weight of the vehicle. The monoalkyl ether of diethylene glycol may be present in an amount of about 0.2% and 25% by weight of the vehicle and the alkanol may be present in an amount between about 5 to 75% by weight of the vehicle. Generally, the alkanol can be present in a hydroalcoholic mixture with water. In certain embodiments, the polyalcohol may be propylene glycol, butylene glycol, hexylene glycol, and ethylene glycol. In certain preferred embodiments, the polyalcohol is propylene glycol.

Alkanols. The alkanol may be an ethanol, isopropanol, or n-propanol. In certain preferred embodiments, the alkanol is ethanol.

Permeation enhancers. In one embodiment, the permeation enhancer contains a saturate fatty alcohol or fatty acid given by the formula CH₃—(CH₂)ₙ—CH₂OH or CH₃—(CH₂)ₙ—(CH)₂COOH respectively, in which n is an integer from 8 to 22, preferably 8 to 12, most preferably 10 or an unsaturated fatty alcohol or fatty acid given by the formula CH₃—(CₙH₂₍ₙ₋ₓ₎)—OH or CH₃—(CₙH₂₍ₙ₋ₓ₎)—COOH respectively in which n is an integer from 8 to 22. Instead of or in addition to the saturated fatty alcohol and fatty acids, second component that is a monoalkyl ether of diethylene glycol. The permeation enhancer of monoalkyl ether of diethylene is, for example, diethylene glycol monoethyl ether or diethylene glycol monomethyl ether. In certain preferred embodiments, the permeation enhancer is diethylene glycol monoethyl ether. The selection of the permeation enhancer can affect the amount and rate of transdermal or transmucosal absorption of the present testosterone formulation. The amount of the permeation enhancer may be optimized. In one preferred embodiment, the permeation enhancer may comprise about 1 to 30% of the formulation by weight.

Gelling Agent. In one aspect of the invention, the present formulation may further include a thickening agent or gelling agent present in an amount sufficient to alter the viscosity of the formulation. A gelling agent can be selected from the group including: carbomer, carboxyethylene or polyacrylic acid such as Carbopol 980 or 940 NF, 981 or 941 NF, 1382 or 1342 NF, 5984 or 934 NF, ETD 2020, 2050, 934P NF, 971P NF, 974P NF, Noveon AA-1 USP; cellulose derivatives such as ethylcellulose, hydroxypropylmethylcellulose (HPMC), ethylhydroxyethylcellulose (EHEC), carboxymethylcellulose (CMC), hydroxypropylcellulose (HPC) (Klucel different grades), hydroxyethylcellulose (HEC) (Natrosol grades), HPMCP 55, Methocel grades; natural gums such as arabic, xanthan, guar gums, alginates; polyvinylpyrrolidone derivatives such as Kollidon grades; and polyoxyethylene polyoxypropylene copolymers such as Lutrol F grades 68, 127. Other gelling agents may include chitosan, polyvinyl alcohols, pectins, and veegum grades.

In certain preferred embodiments, the gelling agent is Lutrol F grades and Carbopol grades. The gelling agent may be present from about 0.2 to about 30.0% w/w depending on the type of polymer. In other preferred embodiments, the gelling agent includes about 0.5%-5% by weight of a thickening agent. The amount of the gelling agent in the formulation may be selected to provide the desired product consistency and/or viscosity to facilitate application to the skin.

Preservatives. The formulation may further include preservatives such as but not limited to benzalkonium chloride and derivatives, benzoic acid, benzyl alcohol and derivatives, bronopol, parabens, centrimide, chlorhexidine, cresol and derivatives, imidurea, phenol, phenoxyethanol, phenylethyl alcohol, phenylmercuric salts, thimerosal, sorbic acid and derivatives. The preservative may be present from about 0.01 to about 10.0% w/w of the formulation depending on the type of compound.

Antioxidants. The formulation may optionally include antioxidants such as but not limited to tocopherol and derivatives, ascorbic acid and derivatives, butylated hydroxyanisole, butylated hydroxytoluene, fumaric acid, malic acid, propyl gallate, metabisulfates and derivatives. The antioxidant may be present from about 0.001 to about 5.0% w/w of the formulation depending on the type of compound.

Buffers. The formulation may further include buffers such as carbonate buffers, citrate buffers, phosphate buffers, acetate buffers, hydrochloric acid, lactic acid, tartric acid, diethylamine, triethylamine, diisopropylamine, aminomethylamine. Although other buffers as known in the art may be included. The buffer may replace up to 100% of the water amount within the formulation.

Humectant. The formulation may further include humectant, such as but not limited to glycerin, propylene, glycol, sorbitol, triacetin. The humectant may be present from about 1 to 10% w/w of the formulation depending on the type of compound.

Sequestering Agent. The formulation may further include a sequestering agent such as edetic acid. The sequestering agent is present from about 0.001 to about 5% w/w of the formulation depending on the type of compound.

Surfactant. The formulation may further include anionic, non-ionic or cationic surfactants. The surfactant may be present from about 0.1% to about 30% w/w depending on the type of compound.

pH Regulator. Optionally, the formulation may include a pH regulator, generally, a neutralizing agent, which can optionally have crosslinking function. By way of example and not limitation, the pH regulator may include a ternary amine such as triethanolamine, tromethamine, tetrahydroxypropylethylendiamine, and a NaOH solution. The pH regulator may be present in the formulations in about 0.05 to about 2% w/w.

Moisturizers and Emollients. Optionally, the formulation may include moisturizers and/or emollients to soften and smooth the skin or to hold and retain moisture. By way of example and not limitation, moisturizers and emollients may include cholesterol, lecithin, light mineral oil, petrolatum, and urea.

For any particular formulation, these other ingredients may be selected to achieve the desired drug delivery profile and the amount of penetration desired. The optimum pH may also be determined and may depend on, for example, the base and degree of flux required.

### Preferred Formulations

### Formulation 1

In one embodiment, the androgen, preferably testosterone, is present in an amount of about 0.50 mg to about 2.4 mg, more preferably, 2.2 mg, in a formulation containing: about 30% to about 98% ethanol or isopropanol; about 0.1% to about 5% isopropyl myristate or isopropyl palmitate; about 1% to about 5% sodium hydroxide; and about 0.1% to about 5% of a gelling agent (weight to weight). In another embodiment, the androgen, preferably testosterone, is present in an amount of about 0.50 mg to about 2.4 mg in a formulation containing: about 50% to about 75% ethanol; about 0.5% to about 2% isopropyl myristate; about 1% to about 3% sodium hydroxide; about 0.5% to about 2% polyacrylic acid; and water in an amount sufficient to make the formulation 100% (weight to weight).

### Formulation 2

In another preferred embodiment, the androgen formulation is substantially free of long-chain fatty alcohols, long-chain fatty acids and long-chain fatty esters in order to avoid potential undesirable odor and irritation effects caused by such compounds during use of the formulation. Thus, advantageously, the formulations of the present invention do not include the undesirable odor-causing and irritation-causing permeation enhancers that were once thought to be necessary for such transdermal or transmucosal formulations.

In this embodiment, the androgen, preferably testosterone, is present in an amount of about 0.50 mg to about 2.4 mg, more preferably, 2.2 mg, in a formulation comprising: a C₂ to C₄ alkanol, a polyalcohol, and a permeation enhancer of monoalkyl ether of diethylene glycol present in an amount sufficient to provide permeation enhancement of the androgen through mammalian dermal or mucosal surfaces. In preferred embodiments, the alkanol is a C₂ to C₄ alcohol selected from the group consisting of ethanol, isopropanol, and n-propanol, the polyalcohol is polypropylene glycol, and the permeation enhancer includes diethylene glycol monomethyl ether, diethylene glycol monoethyl ether, and mixtures thereof.

In preferred embodiments, the formulation comprises 1% testosterone; 47.5% ethanol; 6% propylene glycol; 5% diethylene glycol monoethyl ether; 1.2% Carbomer, Carbopol 980; 0.35% triethanolamine; 0.06% edetate disodium; and 38.89% water. The viscosity of the transdermal gel formulation was 22,000-25,000 cps (Range 16,000-40,000) having a slightly acidic pH, between 5 and 7.

### Formulation 3

In another embodiment, the androgen, preferably testosterone, is present in an amount of about 0.50 mg to about 2.4 mg, more preferably, 2.2 mg, in a formulation containing: a fatty acid percutaneous absorption promoter in an amount of between 0.1% and 20% (w/w), preferably capric acid, lauric acid, myristic acid, palmitic acid, stearic acid, oleic acid, palmitoleic acid, linoleic acid or linolenic acid, ethanol or isopropanol between 10% and 90% (w/w), and a stabilizer comprising the fatty acid ester of the fatty acid and the alcohol and present in an amount of between 0.1% and 10% (w/w), preferably ethyl oleate, isopropyl oleate, isopropyl myristate, isopropyl palmitate, ethyl octanoate, ethyl dodecanoate, ethyl linoleate or ethyl linolenate.

This formulation may further comprise between 0.5% and 20% of a polyalcohol (w/w), preferably glycerol, propylene glycol, polyethylene glycol, or mixtures thereof. The androgen formulation may further comprise between 0.2% and 30% (w/w) of a gelling agent. In preferred embodiments, the gelling agent is selected from carbomers, non-preneutralized acrylic polymers, cellulose derivatives, poloxamers, poloxamines, chitosan, dextran pectins, natural gums, and mixtures thereof. The formulation may also include a neutralizer. In preferred embodiments, the neutralizer is selected from triethanolamine, sodium hydroxide, ammonium hydroxide, potassium hydroxide, arginine, aminomethylpropanol and tromethamine, and mixtures thereof.

### Other Formulations

In certain other formulations, the permeation enhancer may be selected from one or more esters of a long chain alkyl para-aminobenzoate, a long chain alkyl dimethyl-para-aminobenzoate, a long chain alkyl cinnamate, a long chain alkyl methoxycinnamate and a long chain alkyl salicylate. In preferred embodiments, the permeation enhancer is one or more esters selected from the group consisting of C₈ to C₁₈ alkyl para-aminobenzoate, C₈ to C₁₈ alkyl dimethyl-para-aminobenzoate, C₈ to C₁₈ alkyl cinnamate, C₈ to C₁₈ alkyl methoxycinnamate and C₈ to C₁₈ alkyl salicylate, and the formulation further comprises an alkanol selected from the group consisting of ethanol and isopropyl alcohol. In certain preferred embodiments, the resulting formulation is applied to the skin by an aerosol, as a spray, and may be applied using a metered spray dispensing device (e.g., see U.S. Pat. Nos 6,818,226 and 6,798,945).

For certain embodiments, the formulation comprises on a weight basis: (a) from about 1 to about 12% octyl salicylate; (b) from about 45 to about 90% of an alkanol selected from the group consisting of ethanol, isopropanol, and a mixture thereof; (c) from about 5 to about 45% water; and (d) from about 0.5 to about 5% of a gelling agent.

In other embodiments, the formulation has pH of about 4 to about 8 and consists essentially of (i) permeation enhancer, preferably oleic acid; (ii) a C₁ to C₄ alkanol; and (iii) a polyalcohol; and iv) a gelling agent. In one embodiment, the alcohol is a member selected from the group consisting of ethanol, propanol, isopropanol and mixtures thereof, and is present from about 5% to about 65% weight to weight of the formulation, more preferably, 10% to about 40% weight to weight of the composition, and even more preferably, 25% to about 35% weight to weight of the formulation. In preferred embodiments, the polyalcohol is ethylene glycol, butylene glycol or propylene glycol, and the gelling agent is selected from Carbopol 1342, Carbopol 940, Klucel and Klucel HF, and is present at about 0.1% to about 10%.

### Transdermal Patches

The transdermal formulations of the present invention may be delivered to postmenopausal women using a transdermal patch. Transdermal patches for transdermal delivery of androgens may be manufactured by conventional techniques used in the art of transdermal drug delivery devices. For instance, androgens and the formulations described herein may be mixed in desired proportions to form a homogeneous mixture and incorporated into a transdermal device. Various techniques are known in the art for making various types of transdermal devices such as adhesive matrix patches and liquid reservoir system (LRS) patches.

Matrix-type transdermal patches are those in which the drug is contained in and released from a polymer matrix. Matrix patches are known in the art of transdermal drug delivery. Examples without limitation, of adhesive matrix transdermal patches are those described or referred to in U.S. Pat. Nos. 5,122,383, 5,460,820 and 6,365,178.

Matrix-type patches for use in the methods herein comprise an occlusive backing that is impermeable to the androgens and defines the face or top surface of the patch and a solid or semisolid matrix layer comprised of a homogeneous blend of the androgen, a polymeric pressure sensitive adhesive carrier, and optionally one or more skin permeation enhancers.

In certain embodiments, the transdermal matrix-type patch is composed of two layers: a soft flexible backing of polyester and a testosterone-containing film of ethylene-vinyl acetate copolymer that contacts the skin surface and modulates the availability of the androgen. A protective liner of fluorocarbon diacrylate or silicone-coated polyester covers the drug film.

Alternatively, the transdermal matrix-type patch may composed of three layers: a soft flexible backing of polyester and a testosterone-containing film of ethylene-vinyl acetate copolymer. The surface of the drug film is partially covered by the third layer: thin and narrow adhesive stripes composed of polyisobutylene and colloidal silicon dioxide.

In still other aspects of the invention, a liquid reservoir system (LRS) type patch which comprises androgen, and other optional ingredients, such as a permeation enhancer, in a carrier vehicle may be used. LRS patches are known in the art of transdermal drug delivery. Examples without limitation, of LRS transdermal patches are those described or referred to in U.S. Pat. Nos. 4,849,224 and 4,983,395.

LRS patches comprise the androgen and a carrier vehicle comprises a fluid of desired viscosity, such as a gel or ointment, which is formulated for confinement in a reservoir having an impermeable backing and a skin contacting permeable membrane, or membrane adhesive laminate providing diffusional contact between the reservoir contents and the skin. For application, a peelable release liner is removed and the patch is attached to the skin surface.

Particular LRS patches useful herein have a flexible backing of polyester/ethylene-vinyl acetate copolymer film, a drug reservoir of testosterone USP and 1.2 mL alcohol USP gelled with hydroxypropyl cellulose, and an ethylene-vinyl acetate copolymer membrane coated with a layer of a polyisobutylene adhesive formulation that controls the rate of release of testosterone from the system. The patch may also contain a protective liner of silicone-coated polyester to cover the adhesive surface.

In a particular embodiment, the LRS patch contains a metallized ethylene-methacrylic acid copolymer/ethylene vinyl acetate backing film; a drug reservoir of testosterone USP, alcohol USP, glycerin USP, glycerol monooleate, methyl laurate, sodium hydroxide NF, to adjust pH, and purified water USP, gelled with carbomer copolymer Type B NF; a permeable polyethylene microporous membrane; and a peripheral layer of acrylic adhesive surrounding the central, active drug delivery area of the patch.

### Metered Dosage Device

A metered dosage device for administration of the androgen formulation may be used in connection with the methods herein. Any metered dosage device capable of dispensing and administering an androgen in a gel or ointment form may be used (e.g., see U.S. Patent Application Publication No. US2006027064).

According to a preferred embodiment, the dispenser unit comprises a pressure-operable pump, such as a finger-operable pump, which is commonly used to dispense lotions. Such pressure-operable pumps are particularly advantageous for dispensing fluid or liquid transdermal formulations for their convenience and ease of use, as they do not require opening or closing the container cap or lid or squeezing the container to dispense the product. Moreover, since the present metered dosage system enables dispensing the fluid medicament without having to open the cover or lid of the container, it minimizes the problems associated with storing a medicament in a conventional lid-cover container, such as oxidation of the active ingredient and contamination or spoilage of the medicament.

Hence, by providing the pressure-operable pump feature that is also capable of dispensing a predetermined amount, the metered dosage device allows an improved way of dispensing a precise amount of fluid medicaments. Where the medicament contains a certain concentration of an androgen, the present metered dosing device can be used to dispense a precise dosage of the androgen. For example, when used in combination with a gel containing 1% testosterone, the present device may be designed to dispense 0.2 g of the transdermal formulation when activated by pressure, e.g., by pressing on the pump, such that about 2 mg of testosterone is dispensed. Thus, the present device may be conveniently used for self-administration of a precise testosterone dosage, and may be designed to provide the preferred dosage amount, such as up to about 2.4 mg of testosterone, according to the present method for testosterone hormone replacement therapy. When smaller doses are to be applied more often, the device can be designed to dispense 1.0 or even 0.5 mg of testosterone upon each actuation. Thus, a four 0.5 mg doses, two 1 mg doses or a single 2 mg dose can be administered to provide the preferred amount of 2 mg of testosterone each day.

In addition to the metered dosage device described above, a sachet with a metered amount of active ingredient or other metered dosage devices can also be used.

The metered dosage device, therefore, provides an improved way of dispensing and administering a drug in a gel or ointment form by enabling easy and convenient administration of the proper dosage, and eliminates the problems of user over- or under-dose that were common in the conventional gel-type drug administration. Even the most inexperience users can follow a treatment regime that requires precise drug dosage administration.

### High Risk for Cardiovascular Events

The methods of the present invention treat women that have a high risk of cardiovascular events. Such women have a minimum of a 2-point cardiovascular disease risk factor as determined by the following point scale: 1) age 60 to less than 70 years (1 point), 2) 70 years or greater (2 points), 3) diabetes mellitus (2 points), 4) presently smoking at least 10 cigarettes/day (or the equivalent, e.g. uses chew tobacco a minimum of 2 hours daily) (1 point), 5) blood pressure (1 point): systolic≧150 mmHg and/or diastolic≧95 mmHg (based on two readings taken at least 30 minutes apart) and/or taking antihypertensive medications (for treatment of hypertension), 6) dyslipidemia (1 point): LDL>160 mg/dl and/or HDL<45 mg/dl and triglycerides>250 mg/dl and/or taking lipid-lowering medication (over-the-counter products are not considered to be acceptable treatment forms), 7) ankle-brachial index<0.6 (2 points), 8) documented history of cardiovascular disease, i.e., myocardial infarction, stroke, hospitalization for unstable angina/acute coronary syndrome, revascularization of the coronary, carotid, or peripheral circulations.

Methods for determining the above-listed cardiovascular disease risk factors are well known to those skilled in the art. A majority of these cardiovascular risk factors may be identified through a review of patient medical histories, patient questionnaires, physical examination, and standard clinical laboratory testing. For instance, assay kits for measuring HDL, LDL/VLDL, triglycerides, cholesterol and glucose levels in blood are routinely used in hospital and clinical settings and are also commercially available for home use (e.g., Abcam, Inc. Cambridge, Mass.).

### Methods of Treatment

In one aspect of the invention, a method for decreasing the risk of a cardiovascular event in a postmenopausal woman at high risk for cardiovascular events, comprising: administering to the woman a formulation comprising a therapeutically effective amount of an androgen, whereby administering the formulation decreases the risk of cardiovascular events in the woman compared to an untreated postmenopausal woman at high risk for cardiovascular events is provided.

In one embodiment, the therapeutically effective amount of an androgen is provided by oral administration. In certain preferred embodiments, the androgen is testosterone or testosterone undecanoate. In other preferred embodiments, the formulation delivers to the woman a nominal daily, therapeutically effective dose of 300 micrograms of testosterone per day.

In one preferred embodiment, the effective amount of the androgen is administered in a transdermal or transmucosal formulation provided in clear, water washable, cool to the touch, quick drying, spreadable and/or non-greasy formulations, such as a gel, or similar fluid formulations such as but not limited to a cream, lotion, ointment, or suspension, which can be applied directly to the skin. These transdermal or transmucosal formulations advantageously deliver serum testosterone concentrations that are not subject to first-pass metabolism and avoiding wide swings in serum testosterone concentrations while reducing skin reactions. In preferred embodiments, the androgen is testosterone.

Transdermal formulations may be applied once daily, or multiple times per day depending upon the condition of the woman, and may be applied topically to any body part, such as the thigh, abdomen, shoulder, and upper arm. In one embodiment, a formulation in the form of a gel is applied to about a 5 inch by 5 inch area of skin. Application may be to alternate areas of the body as applications alternate. For example, the gel may be applied to the thigh for the first application, the upper arm for the second application, and back to the thigh for the third application. This may be advantageous in alleviating any sensitivity of the skin to repeated exposure to components of the formulation.

Transdermal formulations, therefore, provides a method of providing a therapeutically effective amount of an androgen, preferably testosterone, in a manner that is not only clinically effective but also user-friendly. Not only does the present method provide an effective dosage of the androgen, e.g., testosterone, but it also enables easy administration and compliance by patients, since transdermal/transmucosal formulations are easy and painless to apply. Furthermore, as the formulation is absorbed into the patient's skin or mucosa, it is “invisible” after application, and therefore much more discreet than the conventional patch products that have been used for transdermal testosterone delivery. Manufacturers will also appreciate that the present formulation is more cost-effective to produce as it does not require extra steps and materials, such as adhesives and fabrics. Accordingly, transdermal formulations provide numerous advantages for the consumer and the manufacturer.

The amount of androgen and dosing schedules necessary to provide a therapeutically effective amount may be monitored by following serum concentrations of testosterone. Methods for measuring the serum levels of such hormones, particularly testosterone, are well known to one of ordinary skill in the art. The serum measures are preferably made when the therapeutically targeted level of steady state has been achieved.

In certain embodiments the decrease in the risk of a cardiovascular event in the treated women compared to the risk of cardiovascular events in untreated women is at least 10%, at least 20%, at least 30%, at least 40%, at least 50%, at least 60%, at least 70%, at least 75%, at least 80%, at least 85%, at least 90%, at least 95%, or at least 99%.

The following Examples are illustrative and are not meant to be limiting.

### EXAMPLE 1

Calculation of Expected Number of Cardiovascular Events in High Risk Postmenopausal Women

The following example calculates the expected number of cardiovascular events in a population of postmenopausal women derived from historical data. The estimate of the expected rate of cardiovascular events has been determined from the event rates noted in the Women's Health Initiative (WHI), a Department of Health and Human Services National Institute of Health-sponsored study of women between 50 and 79 years of age, a similar set of women between that age were enrolled for determining the actual number of cardiovascular events as described in Example 2.

Table 1a below is adapted from Table 2 of the Hsia et al (Hsia, J., et al. Arch Intern Med. 2006;166(3):357-65.) report of the primary cardiovascular outcomes observed in the WHI and from the review of the WHI cardiovascular outcomes from Hodis and Mack. (Hodis, H. N. and Mack, W. J. Menopause. 2007) The rates in the table below represent the cardiovascular event rate by age group in the placebo-treated subjects in the combined estrogen/progestin arm of the WHI.

TABLE 1a
Cardiovascular endpoints observed in the placebo arm
of the estrogen/progestin sub-study of the WHI

| | Cardiovascular | | | |
|---|---|---|---|---|
| | death/MI/CABG/ | | | |
| | angioplasty/hospitalized | Stroke | Venous Thrombotic | Total |
| Ages | angina (%) * | (%) ** | Events (%) *** | (%) |
| 50-59 | 0.68 | 0.10 | 0.12 | 0.90 |
| 60-69 | 1.32 | 0.23 | 0.25 | 1.80 |
| 70-79 | 1.84 | 0.48 | 0.31 | 2.63 |

Cardiovascular event rate annualized percent reported in each age group.
* Adapted from Hsia et al, 2006 (Table 2)⁵⁰
** Hodis and Mack, 2007 (Table 5)
*** Hodis and Mack, 2007 (Table 9)

Subjects in the clinical trial described in Example 2 were at somewhat greater risk than those women who were part of the WHI. Rossouw et al describe the risk factors of WHI subjects as “levels of cardiovascular risk factors were consistent with a generally healthy population of postmenopausal women”. (Rossouw, J. E., Jama. 2002;288(3):321-33.) The cardiovascular risk factors in the WHI population were reported by Rossouw et al as follows: baseline use of statins, 6.6%; current smokers, 10.5%; treated diabetics, 4.4%; aspirin use (≧80 mg/d), 19.1%; and hypertensive treatment or BP≧140/90, 35.7%. Table lb below shows the difference in overall cardiovascular event rates in subjects with and without risk factors in the WHI.

TABLE 1b
Overall cardiovascular event rate (annualized percent) in subjects
with and without risk factors in subgroups in the WHI

| | | | Increase risk (fold |
|---|---|---|---|
| | Without (%)* | With (%)* | increase) |
| Smoker | .51 | .91 | 1.8 |
| Hypertension | .37 | .79 | 2.1 |
| Diabetes | .47 | 1.41 | 3.0 |
| Cholesterol meds | .47 | 1.16 | 2.5 |

*Annualized percent reported. Adapted from Hsai et al, 2006 (Table 3)

Overall, the cardiovascular event rate in women in the WHI study who had at least one cardiovascular risk factor was approximately 2-fold greater than in women without any cardiovascular risk factors. In estimating the cardiovascular event rate in this study, it is assumed that the quality of medical care will be slightly improved, overall, over the 10 years since the WHI subjects were enrolled. Thus, a conservative estimate of the relative increase in cardiovascular event rates compared to the overall WHI event rate is approximately 40% higher than that reported in the original publication (i.e., approximately 1.4-fold greater) (Table 1c).

TABLE 1c
Cardiovascular event rate by age in women with a cardiovascular
event rate 1.4 times greater than the WHI

| Age | WHI Rate (%) | WHI Rate × 1.4 |
|---|---|---|
| 50-59 | 0.90 | 1.26 |
| 60-69 | 1.80 | 2.52 |
| 70-79 | 2.63 | 3.68 |

The sample size used to determine the fraction of accrual in each age category in the proposed study was: 50-59, 50%; 60-69, 35%; 70-79, 15%.

The weighted average of cardiovascular events per 1,000 subjects followed on average for 12 months with the fractional accrual by age as described would be as shown in Table 1d below.

TABLE 1d
The estimate of the number of cardiovascular events
per 1,000 subjects per year in the proposed study

| | Fraction of total | Estimated Event Rate |
|---|---|---|
| Age | study (%) | per 1,000 subjects |
| 50-59 | 50 | 6.3 |
| 60-69 | 35 | 8.8 |
| 70-79 | 15 | 5.5 |
| Total | 100 | 20.6 |

Based on an estimated rate of accrual between 150 and 300 subjects per month, the expected composite cardiovascular event rate is 2.0%.

### EXAMPLE 2

Topical Administration of a Testosterone Formulation to Postmenopausal Women Decreases the Risk of Having a Cardiovascular Event.

The following example describes the results obtained from an experimental study of 3,656 postmenopausal women to determine the observed number of cardiovascular events in women at high risk for cardiovascular events that have been administered a therapeutically effective amount of testosterone. Women between the age of 50 and 79 years were enrolled in this experimental study.

A randomized, double-blind, placebo-controlled, multi-center Phase 3 study of the long-term safety and efficacy of a preferred androgen formulation of the present invention in postmenopausal women was conducted. A total of 3,656 women were screened and enrolled in the Phase 3 trial based on predetermined eligibility criteria. Eligible postmenopausal women were at least 50 years of age, have increased risk for cardiovascular disease based on defined cardiovascular risk factors.

The study consisted of a screening period of up to 8 weeks and a 60-month treatment period. Eligible women were randomized in a 1:1 ratio to receive either a testosterone gel (0.22 g; 300 mcg/day) or an equivalent weight of placebo gel (0.22 g) daily. Adverse cardiovascular and breast cancer events (ABC events) were assessed at each subject contact in addition to the usual assessments of safety and concomitant medications. The primary safety outcome measure of the study was the combined occurrence of a subject's first cardiovascular event that includes cardiovascular death, nonfatal stroke, nonfatal myocardial infarction, hospitalized unstable angina, coronary revascularization, and venous thromboembolic events (pulmonary embolism and deep vein thromboses). The co-primary safety outcome measure was the rate of a subject's first invasive breast cancer. The secondary safety outcomes included specific cardiovascular morbidities including cardiovascular death, nonfatal stroke, nonfatal myocardial infarction, hospitalized unstable angina, coronary revascularization, venous thromboembolic events, and all-cause mortality. An additional secondary safety outcome includes breast carcinoma in situ. Cardiovascular events occurring during the course of the study were adjudicated by the Cardiovascular Endpoints Committee. Breast cancer adverse events were reviewed by the Breast Endpoints Committee and include breast cancer and carcinoma in situ. In addition, all breast biopsy pathology reports were reviewed by the Breast Endpoints Committee.

There were eight adjudicated cardiovascular events determined from this Phase 3 clinical trial after greater than 4,000 woman-years of therapy. A comparison of the observed number of cardiovascular events to the expected rate of cardiovascular events revealed that the number of observed events was only about 29% of those expected, resulting in a 71% reduction in cardiovascular events.

A greater number of observed cardiovascular events in the untreated postmenopausal women group reveals that administering a therapeutically effective amount of an androgen, i.e., testosterone, decreases in the risk of having a cardiovascular event in postmenopausal women at high risk for cardiovascular disease.

We claim:

1. A method for decreasing the risk of a cardiovascular event in a postmenopausal woman at high risk for cardiovascular events, comprising: administering to the woman a formulation comprising a therapeutically effective amount of an androgen, whereby administering the formulation decreases the risk of cardiovascular events in the woman compared to an untreated postmenopausal woman at high risk for cardiovascular events.

2. The method according to claim 1, wherein the androgen is selected from the group consisting of testosterone (17-β-hydroxyandrostenone), testosterone enanthate, testosterone propionate, testosterone decanoate, testosterone cypionate, methyl testosterone, testolactone, oxymetholone, fluoxymesterone and enanthate, propionate, cypionate, phenylacetate, acetate, isobutyrate, buciclate, heptanoate, decanoate, undecanoate, caprate and isocaprate esters of testosterone and 4-dihydrotestosterone.

3. The method accordingly to claim 2, wherein the androgen is testosterone.

4. The method according to claim 3, wherein administering the formulation delivers to the woman 300 micrograms of testosterone per day.

5. The method according to claim 2, wherein the formulation is administered by oral administration, transmucosal administration or transdermal administration.

6. The method according to claim 5, wherein the formulation is administered once-per-day.

7. The method according to claim 5, wherein the formulation is a transdermal formulation that comprises an amount of testosterone between about 0.50 mg to about 2.4 mg.

8. The method according to claim 7, wherein transdermal formulation comprises an amount of testosterone between about 2.0 mg to about 2.4 mg.

9. The method according to claim 8, wherein transdermal formulation comprises 2.2 mg of testosterone.

10. The method according to claim 9, wherein the transdermal formulation is provided in the form of a gel, lotion, cream, ointment, emulsion, or suspension.

11. The method according to claim 9, wherein the transdermal formulation comprises at least one of a polyalcohol, alkanol, permeation enhancer, gelling agent, neutralizing agent, buffering agent, moisturizing agent, humectant, surfactant, antioxidant, emollient, or buffer.

12. The method according to claim 11, wherein the transdermal formulation comprises about 30% to about 98% ethanol or isopropanol; about 0.1% to about 5% isopropyl myristate or isopropyl palmitate; about 1% to about 5% sodium hydroxide; and about 0.1% to about 5% of a gelling agent (by weight of the formulation).

13. The method according to claim 12, wherein the transdermal formulation comprises about 50% to about 75% ethanol; about 0.5% to about 2% isopropyl myristate or isopropyl palmitate; about 1% to about 3% sodium hydroxide; about 0.5% to about 2% polyacrylic acid; and water in an amount sufficient to make the formulation 100% (by weight of the formulation).

14. The method according to claim 11, wherein the transdermal formulation comprises, an alkanol in an amount between about 5 to 80%, a polyalcohol in an amount between about 1% to 30%, and a permeation enhancer in an amount between about 1 to 30% (by weight of the formulation).

15. The method according to claim 14, wherein the alkanol is provided in combination with water to form a hydroalcoholic mixture, with the alkanol comprising about 5% to 80% by weight of the mixture and the water comprising about 20% to 95% by weight of the mixture, and the hydroalcoholic mixture is present in an amount of about 40 to 98% by weight of the formulation.

16. The method according to claim 14, wherein the alkanol is a C₂ to C₄ alcohol selected from the group consisting of ethanol, isopropanol, and n-propanol, the polyalcohol is polypropylene glycol, and the permeation enhancer includes diethylene glycol monomethyl ether, diethylene glycol monoethyl ether, and mixtures thereof.

17. The method according to claim 11, wherein the transdermal formulation comprises between 0.1% and 20% of a fatty acid percutaneous absorption promoter (w/w), between 10% and 90% ethanol or isopropanol (w/w), and a stabilizer comprising the fatty acid ester of the fatty acid and the alcohol and present in an amount of between 0.1% and 10% (w/w).

18. The method according to claim 17, wherein the fatty acid percutaneous absorption promoter is selected from the group consisting of: capric acid, lauric acid, myristic acid, palmitic acid, stearic acid, oleic acid, palmitoleic acid, linoleic acid and linolenic acid.

19. The method according to claim 17, wherein the fatty acid ester of the fatty acid is selected from the group consisting of: ethyl oleate, isopropyl oleate, isopropyl myristate, isopropyl palmitate, ethyl octanoate, ethyl dodecanoate, ethyl linoleate and ethyl linolenate.

20. The method according to claim 11, wherein the permeation enhancer is one or more esters selected from the group consisting of a long chain alkyl para-aminobenzoate, long chain alkyl dimethyl-para-aminobenzoate, long chain alkyl cinnamate, long chain alkyl methoxycinnamate and long chain alkyl salicylate.

21. The method according to claim 20, wherein the permeation enhancer is one or more esters selected from the group consisting of C₈ to C₁₈ alkyl para-aminobenzoate, C₈ to C₁₈ alkyl dimethyl-para-aminobenzoate, C₈ to C₁₈ alkyl cinnamate, C₈ to C₁₈ alkyl methoxycinnamate and C₈ to C₁₈ alkyl salicylate.

22. The method according to claim 21, wherein the transdermal formulation further 1. comprises an alkanol selected from the group consisting of ethanol and isopropyl alcohol.

23. The method according to claim 22, wherein the formulation is applied to the skin of the women by an aerosol, as a spray.

24. The method according to claim 11, wherein the permeation enhancer is oleic acid present from about 0.1% to about 10% weight to weight; about 5% to about 65% weight to weight; the alkanol is selected from the group consisting of ethanol, propanol, isopropanol and mixtures thereof present from about 5% to about 65% weight to weight;
the polyalcohol is selected from the group consisting of ethylene glycol, butylene glycol or propylene glycol, and the gelling agent is selected from Carbopol 1342, Carbopol 940, Klucel and Klucel HF present at about 0.1% to about 10% weight to weight.

25. The method according to claim 9, further comprising using a transdermal patch to deliver the transdermal formulation.

26. The method according to claim 6, which further comprises accurately controlling the administration of testosterone by dispensing the formulation from a metered dosage device.

27. The method according to claim 26, wherein the metered dosage device dispenses a precise amount of testosterone for self administration upon a transdermal or transmucosal surface of the subject.

28. The method according to claim 27, wherein the metered dosage device dispenses an amount of 0.22 gram of the transdermal formulation comprising 2.2 mg of testosterone.

29. The method according to claim 1, wherein the woman is surgically postmenopausal or naturally postmenopausal.

30. The method according to claim 28, wherein the woman is surgically postmenopausal or naturally postmenopausal.

31. The method according to claim 4, wherein the decrease in the risk of a cardiovascular event in the treated women compared to the risk of cardiovascular events in untreated women is at least 70.

32. The method according to claim 28, wherein the decrease in the risk of a cardiovascular event in the treated women compared to the risk of cardiovascular events in untreated women is at least 70%.
